(19) United States
(12) Patent Application Publication
(10) Pub. No.: US 2003/0067957 A1
(43) Pub. Date: Apr. 10, 2003

(54) Temperature detective structure of ear thermometer

(76) Inventors: Kuan Yuan Ko, Chubei City (TW); Tao Cheng Lu, Chubei City (JP)

Correspondence Address: ROSENBERG, KLEIN & LEE, 3458 ELLICOTT CENTER DRIVE-SUITE 101, ELLICOTT CITY, MD, 21043, US

(21) Appl. No.: 09/970,859

(22) Filed: Oct. 5, 2001

(51) Int. Cl. G01J 5/02; A61B 5/00

(52) U.S. Cl. 374/130; 600/549; 600/474

(57) ABSTRACT

The present invention provides a temperature detective structure of an ear thermometer, wherein a through receiving space is formed at the center of a fixing seat, and a radiation sensor is disposed at the top inside the receiving space. A heat-spreading sheet is fixedly disposed at the bottom end of the radiation sensor to fix the radiation sensor. A plurality of fixing ribs are annularly disposed on the inner wall of a shell cover to retain the fixing seat in the shell cover. A funneled opening is formed at the top of the shell cover to expose the radiation sensor. The present invention has good sensing effect and a low manufacturing cost.

### FIELD OF THE INVENTION

The present invention relates to an improved structure of an ear thermometer and, more particularly, to a temperature detective structure of an ear thermometer.

### BACKGROUND OF THE INVENTION

Radiation thermometers have been widely used for quick and accurate measurement of the body temperature during a general diagnosis process. Among all body portions used to measure the body temperature, the temperature of the eardrum can much more represent the temperature inside the body than the mouth cavity, the rectum, or the armpit. Use of an infrared ear thermometer is achieved by sticking a temperature probe at the front end thereof deeply into the ear canal to measure infrared radiation emitted from the eardrum in the ear canal, hence obtaining an accurate temperature of the human body.

As shown in FIG. 1, a temperature probe 10 of a prior art ear thermometer 10 comprises a fixing seat 12 with an infrared waveguide tube 16 disposed therein. The top of the waveguide tube 16 has an opening 14, and the bottom thereof has a thermal radiation sensor 18. A shell cover 20 is sleeved onto the outside of the fixing seat 12. A slot 22 forms on the top of the shell cover 20 so that infrared radiation received from the slot 22 via the opening 14 can be transferred to the thermal sensor 18 by the waveguide tube 16, thereby obtaining the temperature in an ear canal.

However, because the thermal radiation sensor 18 of the thermal probe 10 of this kind of ear thermometer is situated below the waveguide tube 16, it is far from the infrared receiving mouth. Infrared radiation received by the thermal radiation sensor 18 needs to be first guided by the waveguide tube 16, thermal gradient thus easily arises, hence letting the thermal radiation sensor 18 be not able to obtain a most direct value of radiation. Therefore, measurement error is easily generated for the temperature measured each time. In consideration of the above problem, the present invention aims to propose a thermal sensor of an ear thermometer.

### SUMMARY OF THE INVENTION

The primary object of the present invention is to propose a thermal sensor of an ear thermometer having good sensing effect so that an exact body temperature can be obtained when measuring the temperature in an ear canal.

Another object of the present invention is to propose a temperature detective structure of an ear thermometer having a reduced cost.

According to the present invention, a through receiving space is formed at the center of a fixing seat, and a radiation sensor is disposed at the top inside the receiving space. A metal heat-spreading sheet is fixedly disposed at the bottom end of the radiation sensor. The radiation sensor is fixed through fixedly retaining the metal heat-spreading sheet in the receiving space. A hollow vertical tube of a bushing seat is disposed in the receiving space to fix the radiation sensor. A plurality of fixing ribs are annularly disposed on the inner wall of a shell cover to fixedly retain the fixing seat in the shell cover. A funneled opening forms at the top of the shell cover.

The various objects and advantages of the present invention will be more readily understood from the following detailed description when read in conjunction with the appended drawing, in which:

### BRIEF DESCRIPTION OF THE DRAWINGS

FIG. 1 is a cross-sectional view of a temperature probe of a prior art ear thermometer;

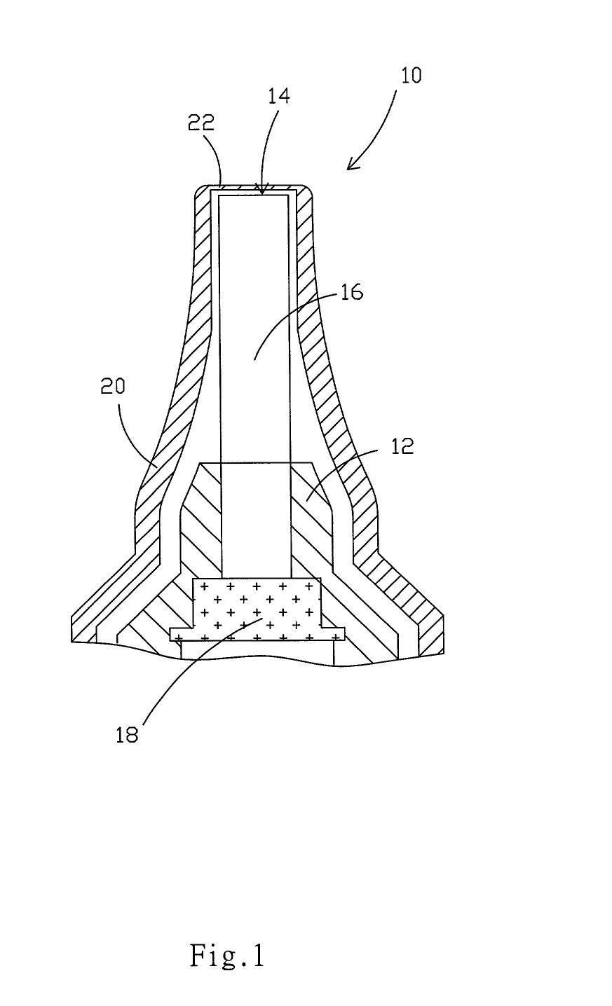

FIG. 2 is a perspective view of the present invention;

FIG. 3 is an exploded perspective view of the present invention;

FIG. 4 is a cross-sectional view of the present invention;

FIG. 5 is a partly cross-sectional view along line A-A′ shown in FIG. 4. and

FIG. 6 is a view of another embodiment of the present invention.

### DETAILED DESCRIPTION OF THE PREFERRED EMBODIMENTS

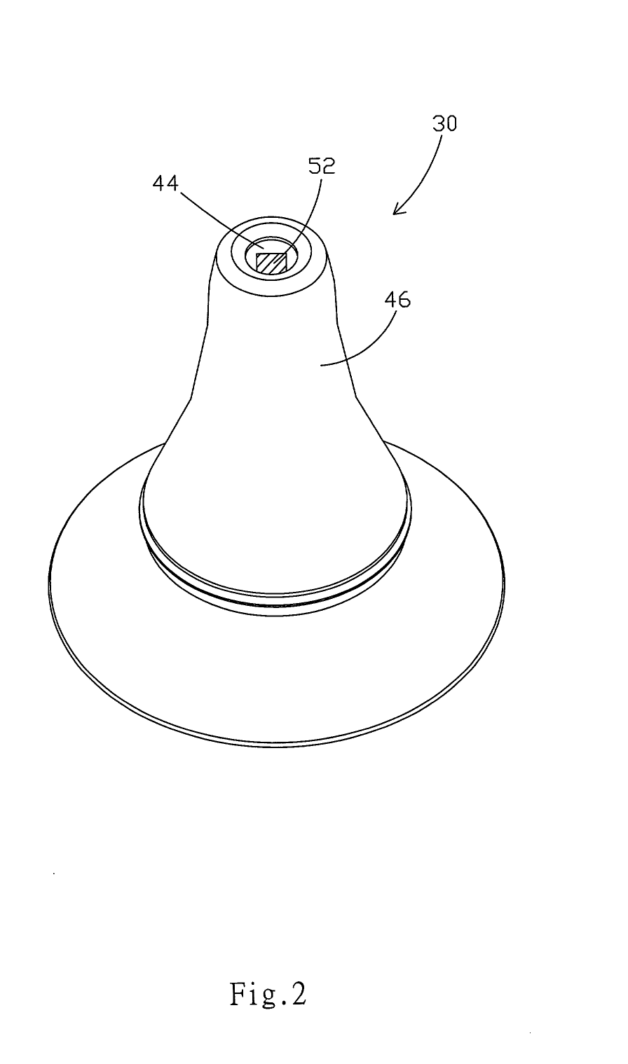

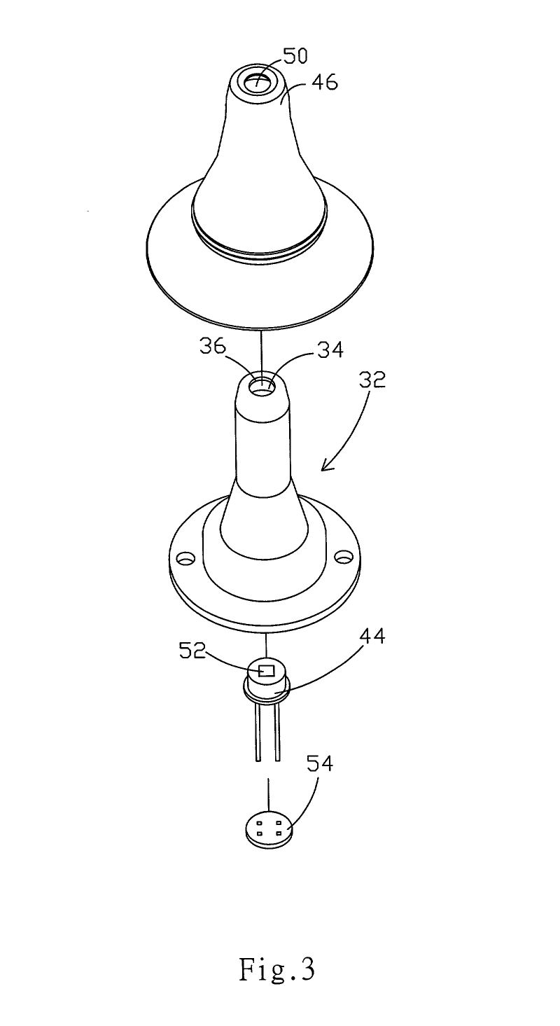

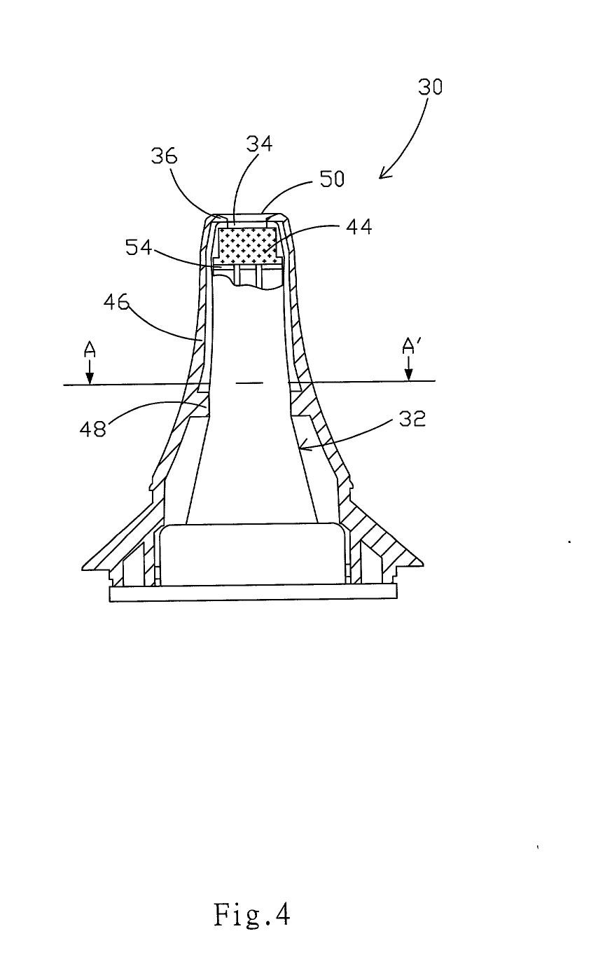

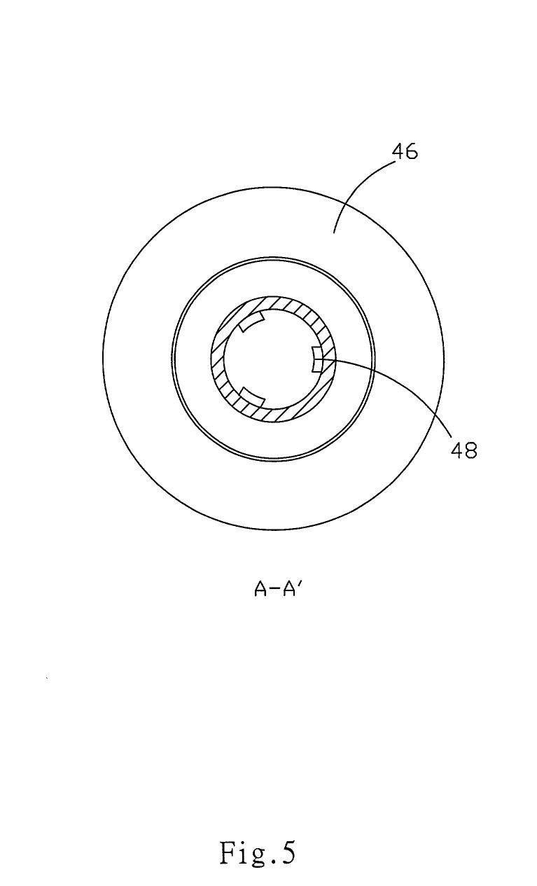

As shown in FIGS. 2 and 3, the present invention comprises a fixing seat 32 with a through receiving space 34 formed at the center thereof. The top edge of the inner wall of the receiving space 34 is bent inwards to form a stop portion 36. A radiation sensor 44 is placed at the top inside the receiving space 34. The outer edge of the radiation sensor 44 is retained with the stop portion 36. A metal heat-spreading heat 54 is disposed at the bottom end of the radiation seneor44, and is fixedly retained in the receiving space 34 to fix the radiation sensor 44. A plurality of fixing ribs 48 are annularly disposed on the inner wall of a shell cover 46 to fixedly retain the fixing seat 32 in the shell cover 46, as shown in FIGS. 4 and 5. An opening 50 is formed at the top of the shell cover 46. The diameter of the opening 50 is the same as that of the top of the receiving space 34 of the fixing seat 32 for exposing the radiation sensor 34 situated in the receiving space 34, thereby facilitating reception of external infrared rays.

The shell cover 46 is made of plastic material. The fixing seat 32 is made of metallic material. The opening 50 of the shell cover 46 forms a funneled structure. The top face of the radiation sensor 44 has a sensing window 52. Infrared radiation enters the radiation sensor 44 via the sensing window 52 so that the radiation sensor 44 can transform temperature increase due to radiation into an electrical output signal, which is then transferred to an electronic measuring circuit (not shown) in the ear thermometer to measure the temperature.

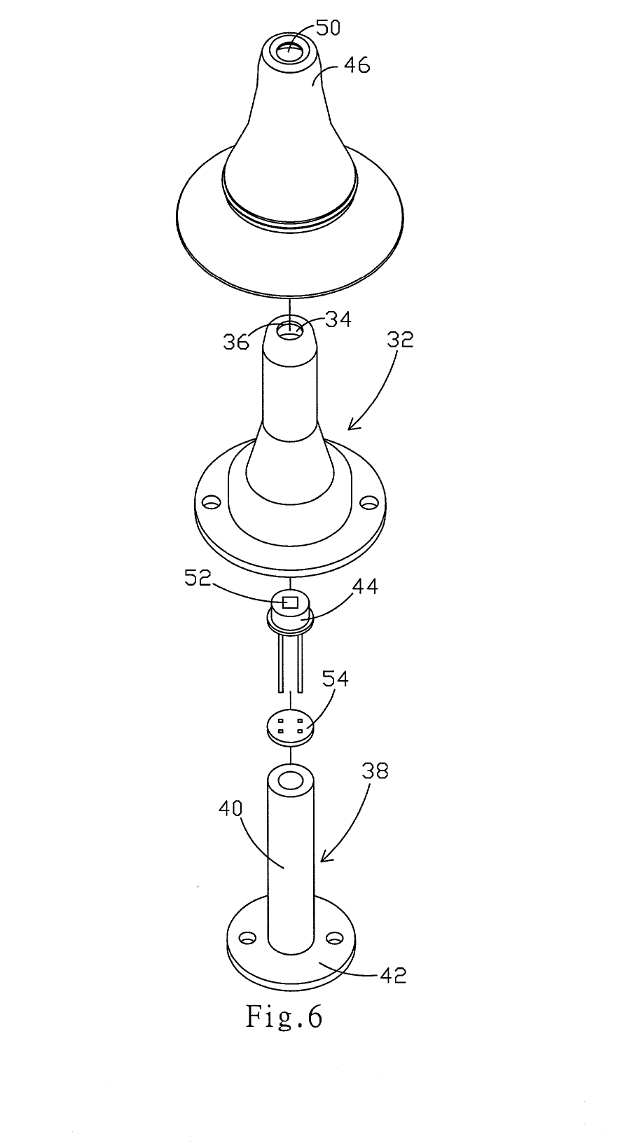

As shown in FIG. 6, a bushing seat 38 comprising a hollow vertical tube 40 and a bottom seat 42 is disposed in the receiving space 34 of the fixing seat 32. The hollow vertical tube 40 is disposed in the receiving space 34 to fix the radiation sensor 44. The bottom seat 42 of the bushing seat 38 is fixedly disposed on the bottom face of the fixing seat 32.

In the present invention, when a temperature detective structure 30 of the ear thermometer is stuck into an ear canal to measure infrared radiation emitted from the eardrum, because the radiation sensor 44 is situated at the front end of the temperature detective structure 30, it can directly sense infrared radiation. As compared to the prior art wherein infrared radiation needs to pass a waveguide tube to reach a sensor, the measuring effect of the present invention is better. Therefore, an accurate body temperature can be obtained. Moreover, in addition to having the function of retaining the fixing seat 32, the structure of the fixing ribs 48 on the inner wall of the shell cover 46 can let gap be kept between the fixing seat 32 and the shell cover 46, hence effectively resolving heat-radiating problem. Additionally, because the fixing seat 32 of the present invention is made of metallic material, the manufacturing and material costs are lower than those of a prior art waveguide tube. Therefore, the present invention has the advantage of reducing the cost.

Although the present invention has been described with reference to the preferred embodiment thereof, it will be understood that the invention is not limited to the details thereof. Various substitutions and modifications have been suggested in the foregoing description, and other will occur to those of ordinary skill in the art. Therefore, all such substitutions and modifications are intended to be embraced within the scope of the invention as defined in the appended claims.

I claim:

1. A temperature detective structure of an ear thermometer, comprising:
a fixing seat with a through receiving space formed at a center thereof;
a radiation sensor placed at a top inside said receiving space, a heat-spreading sheet being fixedly disposed at a bottom end of said radiation sensor and fixedly retained in said receiving space; and
a shell cover with a plurality of fixing ribs annularly disposed on an inner wall thereof to fixedly retain said fixing seat therein, an opening being formed at a top of said shell cover to expose said radiation sensor.

2. The temperature detective structure of an ear thermometer as claimed in claim 1, wherein said opening is a funneled structure so that said radiation sensor can shore up said funneled structure.

3. The temperature detective structure of an ear thermometer as claimed in claim 1, wherein said shell cover is made of plastic material.

4. The temperature detective structure of an ear thermometer as claimed in claim 1, wherein said fixing seat is made of metallic material.

5. The temperature detective structure of an ear thermometer as claimed in claim 1, wherein a bushing seat is disposed in said receiving space to fix said radiation sensor..

6. The temperature detective structure of an ear thermometer as claimed in claim 1, wherein a top edge of an inner wall of said receiving space is bent inwards to form a stop portion for retaining said radiation sensor.
